(19) United States
(12) Patent Application Publication
(10) Pub. No.: US 2011/0218474 A1
(43) Pub. Date: Sep. 8, 2011

(54) APPARATUS FOR THE DESTRUCTION OF TUMOR CELLS OR PATHOGENS IN THE BLOOD STREAM

(75) Inventors: Axel Erich Theuer, Kirchberg (DE); Gerhard Franz Walter, Kassel (DE)

(73) Assignee: ONCOWAVE MEDICAL GMBH, Würzburg (DE)

(21) Appl. No.: 13/059,558

(22) PCT Filed: Aug. 19, 2009
(86) PCT No.: PCT/EP09/06013

§ 371 Date: May 16, 2011

(30) Foreign Application Priority Data
Aug. 19, 2008 (DE) 10 2008 038 309.0

(51) Int. Cl. A61M 1/00

(52) U.S. Cl. 604/4.01

(57) ABSTRACT

The invention relates to an apparatus for destroying tumor cells or pathogens in the blood stream, comprising at least one ultrasonic frequency generator (11) and a device for forming an extracorporeal blood circulation (1) which encompasses at least one heat exchanger (3), at least one blood circulation pump (2, 2′), and a treatment vessel (6) for holding blood. The treatment vessel (6) is mounted downstream of the heat exchanger (3), forms a treatment chamber (13), and is connected to at least one ultrasonic transducer head (8) which is coupled to the ultrasonic frequency generator (11) such that a low-frequency ultrasonic vibration can be introduced into the treatment chamber (13).

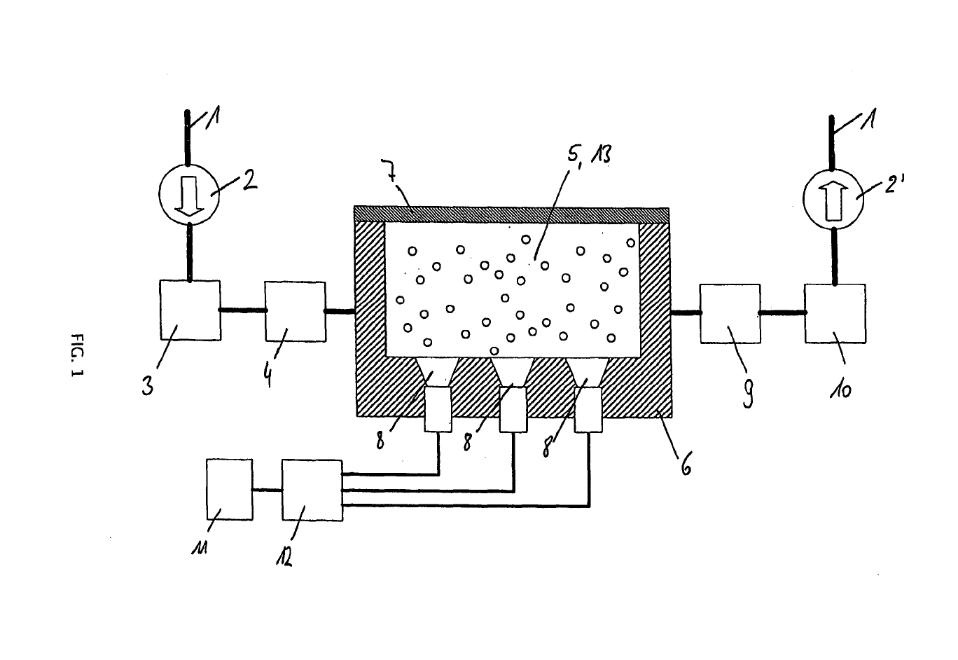

The invention relates to an apparatus for the destruction of tumor cells or pathogens in the blood stream in accordance with claim 1 having at least one ultrasonic frequency generator and a device for forming an extracorporeal blood circulation.

Ultrasonic devices for the therapeutic treatment of tumour diseases are known from the prior art, for instance from U.S. Pat. No. 6,685,657 B2. Such devices are designed such that a high-frequency ultrasonic vibration is applied to the tissue to be destroyed in order to achieve local heating of the tissue which leads to thermal destruction of the tumour cells or pathogens in the blood stream. In order to reduce the thermal effect on healthy tissue, the known apparatus according to U.S. Pat. No. 6,685,657 B2 is disposed at the tip of a catheter such that the ultrasonic transducer head can be brought into direct contact with the tissue to be destroyed. This has the drawback that it is only possible to treat firm tissue or contiguous areas of tissue. Treatment of tumour cells or pathogens in the blood stream, which may lead to propagation of the tumour tissue via the blood circulation, is not possible using the known apparatuses since on one hand it is not possible to establish any direct contact with the tumour cells in the blood and on the other hand it is not possible to rule out damage to healthy blood cells.

Thus the object of the invention is to specify an apparatus for the destruction of tumor cells or pathogens in the blood stream which enables selective destruction of the tumour cells or pathogens in the blood stream and prevents an adverse effect on or damage to healthy blood cells.

According to the invention, this object is achieved by the subject-matter of claim 1.

The invention is therefore based on the idea of specifying an apparatus for destroying tumor cells or pathogens in the blood stream, comprising at least one ultrasonic frequency generator and a device for forming an extracorporeal blood circulation which encompasses at least one heat exchanger, at least one blood circulation pump, and a treatment vessel for holding blood wherein the treatment vessel is mounted downstream of the heat exchanger, forms a treatment chamber, and is connected to at least one ultrasonic transducer head which is coupled to the ultrasonic frequency generator such that a low-frequency ultrasonic vibration can be introduced into the treatment chamber.

The invention has a plurality of advantages, which emerge in particular from a combination of the extracorporeal blood circulation with the low-frequency ultrasonic vibration and the heat exchanger. By means of the extracorporeal blood circulation it is possible for minimally invasive treatment of tumour cells or pathogens in the blood stream to be carried out by conveying the blood from the patient to the treatment vessel and from there back to the patient, i.e. for the low-frequency ultrasonic vibrations to act on the blood outside the body thus preventing damage to the surrounding body tissue.

A further advantage emerges from using the low-frequency ultrasonic vibration which, unlike the high-frequency ultrasonic vibration known from the prior art, essentially brings about no thermal effects within the blood to be treated by which means damage to healthy cells is also prevented.

Malignant cells or tumour cells generally show a different vibration behaviour to that of healthy cells. This is mainly attributable to an altered cytoskeleton of the tumour cells compared to healthy cells. As a result, the resonant frequencies of the tumour cells also differ from those of healthy cells whereby the resonant frequencies usually lie in the low-frequency ultrasonic range. In the apparatus according to the invention, the ultrasonic frequency generator is therefore adapted in such a manner that it is possible to generate a low-frequency ultrasonic vibration that stimulates the tumour cells to vibrate at the relevant resonant frequency such that mechanical destruction of the cells takes place. As the resonant frequencies differ with respect to the tumour and the patient, it is usual to determine the resonant frequencies of the tumour cells to be treated by means of a biopsy and subsequent vibrational analysis prior to treatment.

It has also been shown that the sensitivity of the tumour cells compared to the healthy cells may be increased by means of heat treatment. Therefore, according to the invention, it is proposed to mount a heat exchanger upstream of the treatment vessel such that the temperature of the blood to be treated may be altered prior to ultrasonic treatment.

A dosing unit is preferably disposed between the heat exchanger and the treatment vessel. The dosing unit enables the addition of microbubbles and/or sonosensitizers unless the latter are administered exclusively systemically, i.e. acting on the entire organism, such that the selective differentiability between tumour cells and healthy cells is further increased. Microbubbles, i.e. tiny bubbles of gas enclosed by a stabilising shell, for example of albumin, fatty acids, sugar or polymers, may be stimulated to vibrate due to the effect of ultrasound and promote selective destruction of the tumour cells in the blood. Alternatively or additionally, the vibration behaviour of the tumour cells may be modified by the intracellular absorption of sonosensitizers such that the differences in vibration behaviour between tumour cells and healthy cells are further increased.

In a preferred embodiment of the apparatus according to the invention, an amplifier is mounted downstream of the ultrasonic frequency generator. In this manner, the ultrasonic energy introduced into the treatment chamber is adjusted in order to achieve efficient destruction of the tumour cells or pathogens in the blood stream.

Moreover, at least one filter unit and/or a further heat exchanger may be mounted downstream of the treatment vessel. In this case, the filter unit enables filtering or removal of the substances introduced by means of the dosing unit, in particular microbubbles. The further heat exchanger is used to adjust the temperature of the blood to the patient's body temperature prior to returning the treated blood to the systemic circulation.

The ultrasonic frequency generator can be used preferably to generate an ultrasonic frequency that is variable within the range from 1 kHz to 200 kHz, in particular 1 kHz to 150 kHz, in particular 5 kHz to 150 kHz, in particular 10 kHz to 150 kHz, in particular 16 kHz to 150 kHz, in particular 20 kHz to 120 kHz, in particular 20 kHz to 80 kHz, in particular 20 kHz to 40 kHz. This frequency range corresponds substantially to the resonant frequency range of tumour cells such that efficient destruction of the tumour cells is achieved by introducing ultrasonic vibrations in this frequency range into the treatment chamber.

The low-frequency ultrasonic vibration that can be generated by the ultrasonic frequency generator is preferably pulsable. In this manner, it is possible to generate vibration patterns that can lead to an increase in efficiency for the destruction of tumour cells or pathogens in the blood stream.

In the following, the invention is explained in greater detail based on an embodiment with reference to the associated schematic drawing. The only FIGURE therein shows a process circuit diagram of the apparatus according to the invention with a cross-section through the treatment vessel of the apparatus.

The apparatus for the destruction of tumour cells or pathogens in the blood stream according to the embodiment explained in the following comprises a device for forming an extracorporeal blood circulation 1 with a treatment vessel 6 for holding blood. Treatment vessel 6 as the central component of extracorporeal blood circulation 1 is mounted downstream of a dosing unit 4. Mounted upstream of dosing unit 4 is a heat exchanger 3 which is mounted downstream of a blood circulation pump 2. On the outlet side, treatment vessel 6 is followed by a filter unit 9, which is mounted upstream of a further heat exchanger 10, whereby a further blood circulation pump 2′ follows further heat exchanger 10.

The blood removed from the systemic circulation accordingly flows through extracorporeal blood circulation 1 in the following sequence:
1. Blood circulation pump 2
2. Heat exchanger 3
3. Dosing unit 4
4. Treatment vessel 6
5. Filter unit 9
6. Further heat exchanger 10
7. Further blood circulation pump 2′

Treatment vessel 6 or the treatment basin comprises a treatment chamber 13 into which the treated blood is released. Disposed underneath treatment chamber 13 are three ultrasonic transducer heads 8 which are directed into treatment chamber 13 in such a way that the ultrasonic vibration generated can be introduced into the blood to be treated. A different number of transducer heads, for example one, two or more than three transducer heads, is possible. Ultrasonic transducer heads 8 are integrated in treatment vessel 6, particularly in the base of treatment vessel 6, whereby treatment vessel 6 has bending-resistant or rigid walls to enable efficient sound propagation of the therapeutic ultrasound in treatment chamber 13.

Ultrasonic transducer heads 8 are also electrically coupled to an amplifier 12, which is mounted downstream of ultrasonic frequency generator 11 and connected thereto. The vibration generated in ultrasonic frequency generator 11 is transferred electrically to amplifier 12, amplified therein and transmitted to ultrasonic transducer heads 8, which convert the electrical vibrations into an ultrasonic signal.

A cover 7, which prevents pretreated blood 5 from cooling down, is provided to seal treatment vessel 6. It is possible for the temperature of pretreated blood 5 inside treatment vessel 6 to be kept constant by a heat exchanger (not illustrated) integrated into treatment vessel 6.

Accordingly, the treatment of tumour cells or pathogens in the blood stream is carried out by pumping the blood out of the patient, in particular the blood discharging from the tumour tissue, using blood pump 2 via a heat exchanger 3, in which the blood is brought up to the desired treatment temperature, into dosing unit 4 where microbubbles and/or sonosensitizers are added to the blood to be treated. Blood 5 pretreated in this way is further routed from dosing unit 4 into treatment vessel 6 in which the actual ultrasonic treatment takes place by means of low-frequency ultrasonic vibrations. Treatment vessel 6 may be a basin or a trough, whereby it must be guaranteed that treatment vessel 6 is hermetically sealed. It is possible that a plurality of treatment vessels 6 are disposed behind one another such that treatment of the blood takes place in a plurality of stages. The capacity of treatment vessel 6 is preferably adapted in such a manner that a maximum 500 ml, in particular between 350 ml and 500 ml, blood is taken from the patient for treatment. For example, when using a total of two treatment vessels 6, each treatment vessel 6 holds 200 ml so that essentially a total 400 ml blood is taken from the patient. Added to this is the volume of blood which is located in the hoses of the apparatus, whereby the hose volume essentially forms a fraction of the total volume of blood removed. Hoses that are as short as possible are used in extracorporeal blood circulation 1 to reduce the hose volume. Due to the relatively low capacity of treatment vessel 6, it is ensured that the quantity of blood removed from the patient does not lead to any adverse health effects for the patient. Thus sufficient volume of blood continues to be available to the patient's systemic circulation in order to ensure adequate perfusion, i.e. blood flow, of the bodily organs.

Ultrasonic transducer heads 8 are integrated directly in the base of treatment vessel 6 such that the ultrasonic vibrations act mechanically directly on pretreated blood 5, particularly on the tumour cells to be treated. Ultrasonic transducer heads 8 may also be integrated in the side walls or cover 7 of treatment vessel 6. Basically, ultrasonic vibrations are coupled directly into pretreated blood 5. Treatment vessel 6 is not caused to vibrate. The tumour cells die off due to the direct ultrasonic treatment. This effect is achieved above all in that ultrasonic frequency generator 11 generates a frequency signal which corresponds to the resonant frequency of the tumour cells to be treated. The signal is converted into a sound signal in ultrasonic transducer heads 8 and introduced into pretreated blood 5. After the treatment, pretreated blood 5 is routed into filter unit 9 where the microbubbles or sonosensitizers previously added are filtered out. It is possible that destroyed tumour cells will also be filtered out of pretreated blood 5 using filter unit 9. The filtered blood is conveyed through further heat exchanger 10, whereby the blood temperature in heat exchanger 10 is adjusted to the patient's body temperature. Delivery of the treated blood to the patient takes place via further blood circulation pump 2′, which is disposed between further heat exchanger 10 and the patient's systemic circulation.

### LIST OF REFERENCE NUMBERS

1 Extracorporeal blood circulation

2, 2′ Blood circulation pump

3, 10 Heat exchanger

4 Dosing unit

5 Pretreated blood

6 Treatment vessel

7 Cover

8 Ultrasonic transducer head

9 Filter unit

11 Ultrasonic frequency generator

12 Amplifier

13 Treatment chamber

1. Apparatus for destroying tumor cells or pathogens in the blood stream, comprising at least one ultrasonic frequency generator (11) and a device for forming an extracorporeal blood circulation (1) which encompasses at least one heat exchanger (3), at least one blood circulation pump (2, 2′), and a treatment vessel (6) for holding blood wherein the treatment vessel (6) is mounted downstream of the heat exchanger (3), forms a treatment chamber (13), and is connected to at least one ultrasonic transducer head (8) which is coupled to the ultrasonic frequency generator (11) such that a low-frequency ultrasonic vibration can be introduced into the treatment chamber (13).

2. Apparatus according to claim 1, wherein a dosing unit (4) is disposed between the heat exchanger (3) and the treatment vessel (6).

3. Apparatus according to claim 1, wherein an amplifier (12) is mounted downstream of the ultrasonic frequency generator (11).

4. Apparatus according to claim 1, wherein at least one filter unit (9) and/or a further heat exchanger (10) is mounted downstream of the treatment vessel (6).

5. Apparatus according to claim 1, wherein the ultrasonic frequency generator (11) can be used to generate an ultrasonic frequency that is variable within the range from 1 kHz to 200 kHz, in particular 1 kHz to 150 kHz, in particular 5 kHz to 150 kHz, in particular 10 kHz to 150 kHz, in particular 16 kHz to 150 kHz, in particular 20 kHz to 120 kHz, in particular 20 kHz to 80 kHz, in particular 20 kHz to 40 kHz.

6. Apparatus according to claim 1, wherein the low-frequency ultrasonic vibration that can be generated by the ultrasonic frequency generator (11) is pulsable.
